(19) United States
(12) Patent Application Publication
(10) Pub. No.: US 2026/0293872 A1
(43) Pub. Date: Oct. 1, 2026

(54) HOOF-BOOT SYSTEM COMPRISING A HOOF BOOT HAVING A SENSOR SYSTEM AND COMPRISING ELECTRONIC HOOF COMPONENTS

(71) Applicant: CONTITECH DEUTSCHLAND GMBH, Hannover (DE)

(72) Inventors: Max Konstantin WERHAHN, Hannover (DE); Peter Michael MARIENFELD, Hannover (DE); Moritz PLINKE, Hannover (DE)

(21) Appl. No.: 19/474,860

(22) PCT Filed: Mar. 27, 2024
(86) PCT No.: PCT/EP2024/058226

§ 371 Date: Oct. 13, 2025

(30) Foreign Application Priority Data
Apr. 13, 2023 (DE) 10 2023 203 348.8

(51) Int. Cl. A01L 7/00; A01K 29/00; A01L 3/02

(52) U.S. Cl. CPC A01L 7/00; A01K 29/005; A01L 3/02

(57) ABSTRACT

The present invention relates to a hoof boot system (15, 25) comprising a hoof boot (15) with at least one sensor system (1) and a hoof boot wall (15c) with a hoof component holder (15h) and an electronic hoof component (25) which is held by the hoof component holder (15h) and is designed to read the sensor system (1) of the hoof boot (15).

The invention relates to a hoof boot system with a hoof boot having a sensor system, preferably with a reaction force measuring plate for determining a flat reaction force distribution when the hoof of a hoofed animal strikes the ground and with an electronic hoof component.

For the gait and health analysis of a horse or other hoofed animal (e.g., camel), it is desirable to measure the reaction forces generated when it strikes the ground, not only at a single point, but with a certain displacement across the entire contact area of the hoof. The measured values should enable conclusions to be drawn about the animal's state of health, e.g., with regard to lameness or strain on the musculoskeletal system.

While there are a whole range of solutions that are available for corresponding tasks in the fields of medical diagnostics, training condition analysis and rehabilitation in humans, the availability of corresponding systems for hoofed animals is limited. There is a commercially available product from Tekscan and other systems with a similar purpose, however these are based on measuring acceleration rather than reaction forces.

GB 2 482 192 B discloses the attachment of force sensors to a horseshoe for such purposes and the local storage of their signals and/or their transmission via a wireless transmitter to a remote receiving and evaluation station.

According to DE 10 2011 016 344A1 , force sensors are used in an elastomer body for a corresponding purpose. The elastomer body, which in turn is to be inserted into a hoof boot, is designed to enable the use of commercially available resistive force sensors with their limited force measuring range for reaction force analysis in horses with their relatively high ground pressure.

From US 2020/319044 A1 we learn of another system for the area-dissipated recording of reaction forces on the hooves of a hoofed animal, which transmits the sensor signals via a wireless transmitter to a remote evaluation unit. This system has a complex design consisting of a base plate and a ground plate, which are guided precisely in relation to each other and between which several force application cylinders and a plurality of resistive force measurement sensors are housed, as well as a microprocessor unit, a communication unit and a battery to supply force to the components. The function of the ‘force application cylinders’ is not apparent from the printed material, nor is the method of attachment to the hoof.

DE 10 2021 211 795.3 (unpublished) describes a reaction force measuring plate for recording the distribution of ground reaction forces across the ground contact area of the foot of a hoofed animal or the foot of a human being when it strikes the ground, with a rigid carrier plate with a first surface facing the ground during use and a second surface facing the hoof or foot, a plurality of flat force measurement sensors fixed in position on the first surface of the carrier plate, and several elastic force transmission studs, especially a plurality corresponding to the plurality of force measurement sensors, fixed to the free surfaces of the force measurement sensors.

A disadvantage of the known possibilities for measuring the force exerted by the foot of a hoofed animal or the foot of a human being when it strikes the ground, i.e., when it comes into contact with the ground, is that only the distributions of force can be measured, however no total forces occur, as shunt forces occur in addition to the discrete sensor positions.

A disadvantage of using individual force transmission studs for each force measurement sensor of the reaction force measuring plate of DE 10 2021 211 795.3 is that even a slight misalignment between the force transmission studs and the force measurement sensor during operation causes the characteristics or calibration curve of the force measurement sensor (measured value vs. applied force) to change. This can result in significant inaccuracies in the measured force.

Another disadvantage is that different hoof shapes and/or ground conditions, for example, can lead to an undefined transmission of force and force dissipation. This can result in shunt forces, which prevent the load from being conducted to the discrete sensor positions as desired.

Another disadvantage is that when used on hoofed animals with iron shoes (e.g., horses), irregularities in the shoeing, e.g., due to protruding nails, can occur, which can greatly influence the distribution of force on a rigid plate. This would not be the case in the reference state without a measuring system on soft ground, as small unevennesses in the ground can sink in and thus be compensated for. An incorrect interpretation of the measurement results could be the case.

Another disadvantage is that the state of the art electronics either have to be connected directly to the horse using complex cabling or only acceleration signals are detected, so that in terms of the design, no special external interfaces to an external sensor need to be provided.

One object of the present invention is to improve the possibilities for using sensors on the hoof and/or the leg of a hoofed animal, especially a horse. The electronics of the sensor system should be especially compact, visually appealing, easy to operate, robust and/or tightly constructed and attached to the hoof boot.

Preferably, its maintenance and dismantling should generally be possible and preferably, as simply and quickly as possible. Additionally or alternatively, the force measurement of the foot of a hoofed animal when it strikes the ground should be improved. Especially, a preferably compact sensor plate with discrete sensor positions and the transmission of force into the same is to be created, resulting in stable and robust sensor characteristics. Additionally or alternatively, the transmission of force without a shunt force should be used to enable the measurement of absolute forces and minimize potential errors in the measured force distribution. In any case, this should be done in a way that is as simple, cost-effective, space-saving and/or weight-saving as possible. At the very least, an alternative to the familiar options should be created.

The object is achieved according to the invention by a hoof boot system, by a hoof boot and by an electronic hoof component having the features of the independent patent claims. Advantageous further embodiments are described in the subclaims.

Thus, the invention relates to a hoof boot system comprising a hoof boot with at least one sensor and a hoof boot wall with a hoof component holder and with an electronic hoof component which is held by the hoof component holder and is designed to read the sensor system of the hoof boot. The electronic hoof component can be designed so that it is non-destructively removable from the hoof component holder, which can facilitate charging, reading and/or replacement in the event of damage. This can be done quickly and easily, for example by means of a snap-in connection or the like, or in a more durable but more complex way, for example by means of screws, in which case, replacement can be a replacement in the event of damage.

Preferably, the electronic hoof component can also be permanently separated from the hoof component holder and can only be separated through destruction, for example, by means of a riveted connection. This can be a particularly robust and durable connection that can withstand the high loads on the foot of a hoofed animal.

The electronic hoof component can, especially enable one-way or unidirectional and/or two-way and/or bidirectional, preferably wireless, communication. The electronic hoof component can especially have a preferably rechargeable electrical energy storage device for supplying and operating the electronic hoof component and preferably, the sensor system as well.

In any case, the electronic hoof component can accommodate electronic components such as data detection units, data formatting units, for example for signal filtering, data processors and/or data transmitters, also known as an ECU (Electronic Control Unit), either as individual components or as combined components, so that these can be used on the hoof but do not have to be arranged below the hoof, thus avoiding corresponding stresses. This also applies to the optional electrical energy storage system already mentioned. A radio module of a data transmitters can protrude from the electronic hoof component, at least by means of an antenna.

In addition to the sensors of the hoof boot, the electronic hoof component can also have additional sensors, such as a 3-axis acceleration sensor, a 3-axis gyroscope and/or a temperature sensor. This allows additional sensor information to be obtained from the hoof.

In any case, the information detected by the sensors of the hoof boot itself and, if applicable, the sensors of the electronic hoof component can also be used to analyze the movement and/or health status of the hoofed animal, as will be described in more detail below using specific sensors.

As a complete system that can be applied to the hoofed animal, up to four such electronic hoof components can be used, which can communicate with each other, especially via radio. One of the electronic hoof components in the system can communicate with a display device such as a smartphone with an app. This electronic hoof component represents the peripheral device to relative to the display device and thus functions as the central electronic hoof component relative to the other electronic hoof components.

This central electronic hoof component can also change during use depending on the operating conditions, in order, for example to always use the electronic hoof component with the best reception strength relative to the receiving unit as a “peripheral”. Alternatively, all electronic hoof components can function as “peripherals” relative to the display device. Optionally, the system's range can be improved using a Bluetooth repeater. The repeater can, for example, be positioned near the head of the hoofed animal.

The hoof boot wall can also include a so-called “upper” of the hoof boot, which can be a textile combination (fabric, leather, plastic, . . . ) that can be applied to the sole.

According to one aspect of the invention, the electronic hoof component is accommodated and held in a securing system, preferably in an interior space, of the hoof component holder. This can enable a secure hold, especially due to the movements of the hoof of the hoofed animal. The hoof component holder can thus exert a sufficient holding force on the accommodated electronic hoof component in order to hold it in such a way, that even in the event of intense movements of the hoof, a relative movement between the electronic hoof component and the hoof component holder, and as such, the hoof boot, can be excluded.

Preferably, the hoof component holder has a protective edge in order to protect a gap between the accommodated electronic hoof component and the hoof component holder from contamination. This can keep out contaminants that could cause this gap to widen, weakening the durability of the fixed connection between the hoof component holder and the accommodated electronic hoof component, as well as enlarging the gap, which could accelerate the weakening.

Designing the protective edge so that it can preferably be folded up elastically can still make it possible to reach the rear wall of the accommodated electronic hoof component in the area of the gap to the hoof component holder, for example in order to guide a bow-shaped charging clip from above over the accommodated electronic hoof component and to reach the corresponding charging contacts on one of the sides or on both sides of the accommodated electronic hoof component.

According to a further aspect of the invention, the hoof component holder has a stop edge along the joining direction, and the electronic hoof component has a corresponding stop edge opposed along the joining direction. This allows the movement of inserting the electronic hoof component into the hoof component holder to be restricted in a defined manner. Such a defined stop can also assist the holding force during movements. This also helps to avoid a gap at this point, into which dirt could penetrate, as previously described.

According to a further aspect of the invention, the hoof component holder has a pressure compensation element perpendicular to the joining direction and/or the electronic hoof component has a pull-out protection device opposed perpendicular to the joining direction.

The pull-out protection device can be used to create a positive connection perpendicular to the joining direction, which can improve the holding force. This can especially ensure a sufficiently secure holding force so that even in the event of intense movements of the hoof, a relative movement between the electronic hoof component and the hoof component holder, and as such, the hoof boot can be excluded. This type of holder and/or connection can alternatively or additionally be arranged in any other position with sufficient space and/or installation space.

The pressure compensation element can be designed as a semi-permeable membrane, which allows the exchanging of air and is simultaneously waterproof. This can prevent negative pressure in the electronics and the “intake” of water.

According to a further aspect of the invention, the electronic hoof component has at least one charging port, preferably a pair of charging contacts. This may make it possible and/or easier to place a charging clip, preferably in the shape of a bow, on the accommodated electronic hoof component in order to electrically recharge the accommodated electronic hoof component.

According to a further aspect of the invention, the electronic hoof component has an operating element, preferably an on/off switch, which is accessible from the outside, preferably laterally and facing away from the hoof during use. This can at least enable simple or elementary operating actions, especially switching on and off, to be carried out directly on the electronic hoof component. Preferably, for example, querying the charge level, coupling a wireless data connection and the like may still be possible.

According to a further aspect of the invention, the electronic hoof component has at least one display element visible from the outside, preferably laterally and facing away from the hoof during use. This can make it possible for at least simple information such as the condition or status directly to be displayed on the electronic hoof component. When using several hoof boot systems according to the invention on a hoofed animal, the respective configured assignment between the hoof component and the hoof can be displayed by means of the display element. For this purpose, four display elements can be used per hoof component, which can be arranged in a suitable manner to one another so that a user can directly and intuitively identify the hoof on which exactly this hoof component is to be used, based on an activated and/or illuminated display element. Accordingly, the hoof component can be attached to the appropriate hoof or configured to the correct hoof so that the assignment of hoof component and hoof is correct, for example in an app.

Additionally or alternatively, further display options such as battery level indication, charging indication during electrical charging, coupling mode of a wireless communication connection and the like may be provided.

According to a further aspect of the invention, the electronic hoof component has four display elements that are visible to the outside, preferably laterally and facing away from the hoof during use. These display elements are arranged and configured to display the respective hoof during use. This can make it easier for the user to attach the respective hoof boot system to the corresponding hoof of the hoofed animal.

According to a further aspect of the invention, the hoof boot has a hoof boot sole which completely encloses the sensor system relative to the ground, and the hoof boot wall is integrally formed with the hoof boot sole and encloses the sensor system and, during use, the hoof, at least in sections, and preferably at least substantially. This can be a simple and at the same time, particularly protective way of implementation.

Preferably, the hoof boot wall has a sole profile facing away from the carrier plate. This can improve surefootedness on the ground.

According to a further aspect of the invention, the electronic hoof component is electrically connected to the sensor system of the reaction force measuring plate in a conductive way by means of a connector element, wherein the connector element passes through a connector element opening of the hoof boot wall, preferably directly above the hoof boot sole and/or laterally. This means that a sensor outlet of the sensor system near the sole base can be guided through an opening into the outer area of the hoof boot. This can protect the connection from stress, especially from striking the ground.

Preferably, the sensor outlet can be guided out of the shoe through an opening in the side wall, which can provide further relief and/or protection compared to arranging the opening at the tip of the hoof. In any case, the sensor outlet can be guided out on the outward-facing side of the boot (right hoof boot, right outlet, left hoof boot, left outlet) to avoid any contact of the connection and especially, the electronic hoof component with the other hoof. In any case, the electronic hoof component can be connected as an electronic unit with the sensor system of the hoof boot and positioned on the side, outside of the hoof boot.

According to a further aspect of the invention, the connector element opening is at least partially, and preferably completely, closed by a connector element clamp. This can prevent or at least make it more difficult for dirt and liquid to enter the interior of the hoof boot in the area between the underside of the hoof and the sensor system at this point. In any case, external influences and/or environmental factors such as dirt, mud, moisture and/or water, stones and the like can be kept away from the sensor system in order to ensure their functionality and/or improve their longevity.

According to a further aspect of the invention, the electronic hoof component is arranged on the outside of the hoof boot wall, wherein the hoof boot wall has a hoof component guard which surrounds the electronic hoof component at least from below, and furthermore, preferably at least on one side, and particularly preferably on both sides. Thus, a holder for the electronic hoof component can be provided, which lies over the cable and partially over the electronics, protects them from damage and/or fixes them to the outer wall of the shoe. Furthermore, a protective edge can be formed from the sole and/or the wall, which at least partially surrounds the holder and thus protects it from impact and dirt.

Alternatively, the holder can be made as part of the sole, which is subsequently folded up and attached to the outer wall of the shoe.

According to a further aspect of the invention, the sensor system is a reaction force measuring plate for detecting the distribution of the ground reaction force over the ground contact surface of the foot of a hoofed animal when it strikes the ground, with a preferably rigid carrier plate with a first surface facing the hoof in use and an opposite second surface facing the ground, or conversely, a plurality of flat force measurement sensors fixed in a fixed position on the first surface of the carrier plate, a plurality of elastic force transmission studs, especially a plurality corresponding to the plurality of force measurement sensors, which are fixed to the free surfaces of the force measurement sensors.

This aspect of the invention is based on the idea of using a reaction force measuring plate as the sensor of the hoof boot to detect the ground reaction force distribution. This measuring device of the hoof boot can be formed with a carrier plate that is essentially both rigid and incompressible (but possibly elastically flexible), to one surface of which a plurality of force measurement sensors are attached, and the other surface of which is placed on the underside of the hoof during use and is in any case in effective contact with the underside of the hoof.

As explicitly stated and described above, the carrier plate can be used with the first surface facing the ground and the second opposite surface facing the hoof. However, the arrangement can also be reversed, with the carrier plate facing the hoof with the first surface in use and facing the ground with the second opposite surface. This can increase the utility and design possibilities.

Especially, the arrangement of the carrier plate with the first surface facing the hoof during use is advantageous because the force measurement sensors and elastic force transmission studs then act against the hoof. Accordingly, the carrier plate does not need to be connected directly to the hoof or horseshoe. Instead, the fixation can be achieved, for example, by means of a hoof boot, which can also be formed by a protective element of the reaction force measuring plate itself, as will be described in more detail below. In any case, the force measurement sensors and elastic force transmission studs can then act towards the underside of the hoof and a cable outlet of the force measurement sensors can be guided outwards from the hoof boot directly above the sole base of the hoof boot and without a relative movement between the cable outlet/clamp and the cable and there, it can be electrically connected to the electronic hoof component.

Furthermore, the invention includes the idea of assigning a force transmission stud adapted to the effective area of the sensor to each of the force measurement sensors on its free surface. In principle, it is also possible that the system is configured using force transmission studs with a larger base area, which are not assigned to a single force sensor but to two or more force measurement sensors together, and which transfer the ground reaction forces that occur into these sensors. In extreme cases, the measuring plate could even have only one single force transmission stud, with a base area covering all of the force measurement sensors, provided that this force measurement stud, due to its structure and mounting on the carrier plate, enables differentiated transmission of force to the various sensors.

For common practical applications, 3 to 8 force measurement sensors, and especially 5 to 7 sensors, are currently considered sufficient. Preferably, at least five force measurement sensors can be used. Preferably, at least, and particularly preferably exactly seven force measurement sensors can be used, which can be arranged along the edge. In any case, the force measurement sensors can be arranged at equal distances from each other in the circumferential direction. This can enable representative recording of force values with limited cost.

Such a reaction force measuring plate has a simple design and is therefore inexpensive to manufacture, robust in practical use, and easy to handle. It also enables a sufficiently resolved recording of the reaction forces across the surface of the hoof with low leakage of the force and therefore high accuracy. It is also flat and lightweight in terms of structure, making it not only easy to use but also easy to transport.

In any case, the corresponding features and advantages can be integrated into a hoof boot. For this purpose, the reaction force measuring plate can be fixedly arranged inside a hoof boot and thus come into contact with the underside of the hoof of the hoofed animal during use.

The sole of the hoof boot can be formed in one piece, i.e., integrally, in a largely semi-oval shape with the side wall of the hoof boot and can position and guide the reaction force measuring plate laterally. The hoof boot wall or collar can preferably comprise at least ¾ of the sole and preferably be formed completely circumferential. In any case, the side wall of the hoof boot, i.e., the hoof boot wall, can have at least the height of the reaction force measuring plate. The sole of the hoof boot, i.e., the hoof boot sole, can preferably be formed largely in a semi-oval shape with one or more bulges to prevent rotation of one or more of the elements of the reaction force measuring plate. This can preferably be done in the form of two “fins” in the heel area.

The invention also relates to a hoof boot for use in a hoof boot system as described above. Thus, a hoof boot can be provided to implement and utilize the properties and advantages described above.

The invention further relates to an electronic hoof component for use in a hoof boot system as described above. Thus, an electronic hoof component can be provided to implement and utilize the features and advantages that were previously described.

According to a further aspect of the invention, the hoof boot further comprises an inner protective element which runs parallel to the carrier plate and encloses at least the sensor system and/or the force measurement sensors relative to the hoof. This allows the sensor system or force measurement sensors to be better protected from contamination and moisture.

According to a further aspect of the invention, the inner protective element has a seal on the edge which seals off the inside of the hoof boot wall. This can improve the protective effect.

According to a further aspect of the invention, the hoof boot further comprises at least, preferably exactly, one rigid load distribution plate which is arranged parallel to the carrier plate and facing away from the carrier plate on the elastic force transmission studs and facing the hoof. Thus, the rigid load distribution plate spans several, to all force measurement sensors and/or the distances between them. This allows transmission of force per force measurement sensor without shunt forces, which can improve the quality of force measurements.

Preferably, the rigid load distribution plate can be made of a material with very high strength, rigidity and good maximum elongation. Polyamide, with an additional reinforcement can be used for this purpose. The rigid load distribution plate can be implemented as an organic sheet metal made of polyamide with glass fiber reinforcement, which is particularly preferred.

According to a further aspect of the invention, the hoof boot further comprises at least one, preferably elastomeric, force transmission surface element, which is fixed in position parallel to the carrier plate and facing away from the first surface on the force measurement sensors, wherein the elastic force transmission studs are fixed on the free surface of the force transmission surface element directly opposite exactly one of the force measurement sensors. The force transmission surface element is also arranged between the carrier plate and the load distribution plate.

This aspect of the present invention is based on the realization that the force transmission studs, which are each arranged directly opposite one of the force measurement sensors, can be used to transmit the ground contact forces to the respective force sensors, as described in DE 10 2021 211 795.3. In addition, however, the fact that a force transmission surface element accommodates the force transmission studs and thus positions them relative to each other and to the force measurement sensors means that any misalignment between the force transmission studs and the force measurement sensor can be avoided during operation, or at least sufficiently reduced in order to avoid and/or at least sufficiently reduce a change and/or an influencing of the characteristics and/or the calibration characteristic curve in order to be able to measure the forces with sufficient accuracy.

The force transmission surface element can also be called a pressure conduit element. In any case, the force transmission surface element can be designed as a thin flat plane, which can preferably be bonded to a carrier film by material engagement, especially by vulcanization, as will be described in more detail below.

The force transmission surface element can also serve as a thin protective layer and, for this purpose, be designed in such a way, that the force transmission surface element covers and protects at least most of the force measurement sensors or their sensor carrier film.

According to a further aspect of the invention, the force transmission studs and/or the force transmission surface element are made of an elastomeric material. This can cause and/or enable elastic force transmission. Especially, the hardness of the elastomer or elastomeric material of the force transmission studs and/or the force transmission surface element can be between 30 and 85 ShA.

According to a further aspect of the invention, the force transmission studs are integrally formed with the force transmission surface element. Thus, the force transmission studs and the force transmission surface element are integrally formed. This can be done using the same material or different materials, although the latter can increase both the manufacturing costs and the design options.

According to a further aspect of the invention, the force transmission studs have a linear or trapezoidal contact surface with the ground and/or with the respective force measurement sensor. Preferably, there is a linear or elongated contact surface on the side facing away from the force measurement sensor and a larger, wider contact surface in preferably, the direction of the force measurement sensor. This can be achieved, for example, by using a trapezoidal shape, but also by using a straight-line design for the height of the force transmission studs. In any case, this can affect force transmission.

According to a further aspect of the invention, the force transmission surface element is bonded to the force transmission studs with the force measurement sensors or a sensor carrier film facing away from the force transmission studs. This can represent a connection option that can be made directly between force measurement sensors and force transmission surface elements. If the force measurement sensors are arranged on a sensor carrier film, and especially, printed on it, as will be described in more detail below, the sensor carrier film can also be covered and protected by means of the force transmission surface element.

According to a further aspect of the invention, the force transmission surface element is vulcanized onto a carrier film away from the force transmission studs, and the carrier film is bonded to the force measurement sensors or a sensor carrier film. The carrier film can thus be used to create a surface that improves the adhesive effect on the force measurement sensors and/or a sensor carrier film. The carrier film may preferably consist of a material chemically similar to the sensor carrier film in order to enable or promote a material bond with the sensor carrier film. In any case, the carrier film may preferably consist of a thermoplastic or elastomeric material (e.g., TPE, NR, EPDM), which can be bonded to the sensor carrier film by material engagement, preferably in a vulcanization process or in an injection molding process.

According to a further aspect of the invention, in geometric configurations adapted for use with various ungulates or other vertebrates (including humans), the carrier plate has the shape of a closed horseshoe, circular ring, U, or a polygon with a recess in the central area.

According to a further aspect of the invention, in a technologically advantageous embodiment, the force measurement sensors, together with associated sensor signal lines and optional power supply lines, are implemented on a continuous sensor carrier film which is fixed to the first surface of the carrier plate.

According to a further aspect of the invention, the force measurement sensors and/or the sensor carrier film are bonded to the carrier plate and/or the force transmission studs are bonded to the force measurement sensors. This can represent an easily realizable embodiment of the force measurement sensors and/or the above-mentioned sensor carrier film with the carrier plate.

According to a further aspect of the invention, the effective area of the force measurement sensors is in the range between 0.5 cm² and 10cm² , and especially between 2 cm² and 5 cm². It is understood that when using a relatively large number of sensors, and especially in configurations of the measuring plate intended for animals with a relatively small hoof area, the effective area may be relatively small, whereas in configurations with a relatively small number of sensors and for animals with large detection areas, it may be closer to, or even above the preferred upper limit.

According to a further aspect of the invention, in the interest of the technologically easy and cost-effective manufacturability of the sensors and configurability of different designs of the measuring plate, all force measurement sensors essentially have the same geometric shape and effective area.

According to a further aspect of the invention, the force measurement sensors are resistive-dielectric sensors, which especially, comprise a first conductive layer, a dielectric layer on top of this, which is surrounded and delimited by a spacer that determines the shape of the force measurement sensor, and a second conductive layer on the dielectric layer and the spacer. In addition to the structure mentioned here as a variant, such resistive force sensors can also have a different, i.e., known, structure. In principle, piezoelectric, capacitive, or inductive sensors or electroactive polymers can also be used in the reaction force measuring plate in addition to resistive dielectric sensors.

According to a further aspect of the invention, material-related embodiments may provide that the carrier plate consists of organic sheet metal, spring steel, or plastic, and/or the force transmission studs consist of elastomer, and/or the load distribution plate consists of organic sheet metal, spring steel, or plastic, preferably polyamide with a strength carrier, preferably a glass fiber reinforcement.

Specifically, the hardness of the elastomer of the force transmission studs can be between 30 and 85 ShA.

The advantages and practicalities of the invention are also apparent from the description of embodiments based on the figures. These show:

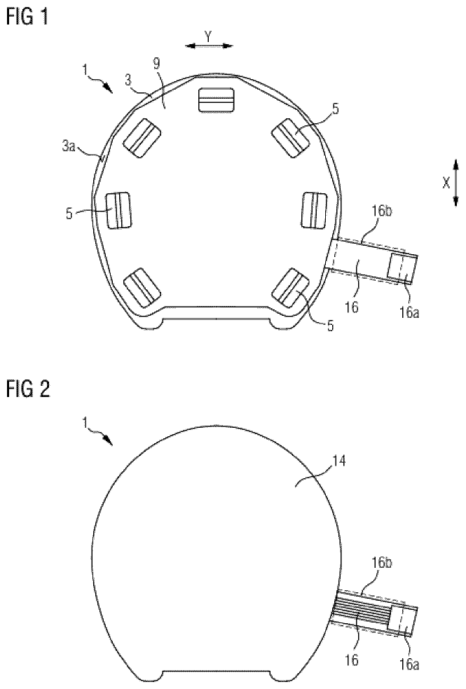

FIG. 1 the structure of an exemplary reaction force measuring plate from a perspective top view of the elastomeric force transmission surface element;

FIG. 2 a top view of the inner protective element of the reaction force measuring plate;

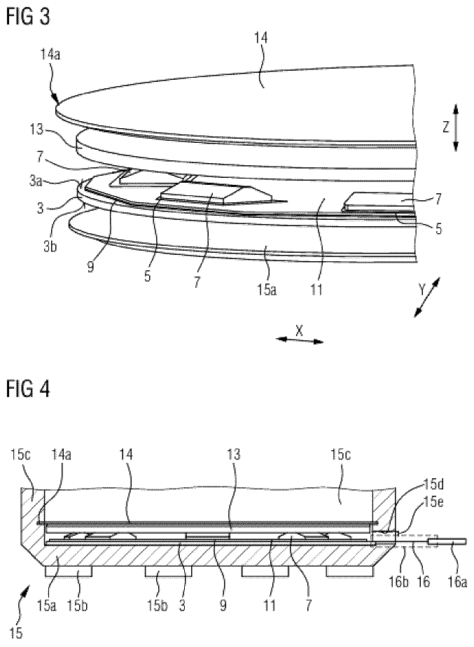

FIG. 3 a perspective view of a section of FIG. 2 as an exploded view with an underside of a hoof boot according to the invention;

FIG. 4 a cross section through the hoof boot with the reaction force measuring plate of FIGS. 1 to 3;

FIG. 5 a side view of the hoof boot including the electronic hoof component;

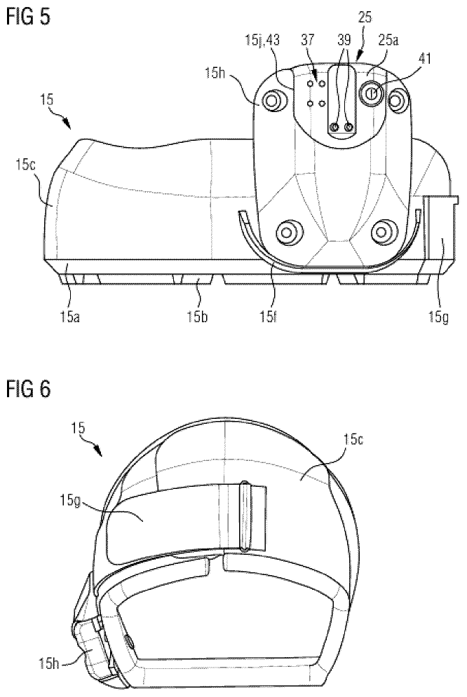

FIG. 6 the representation of FIG. 5 directly from the front;

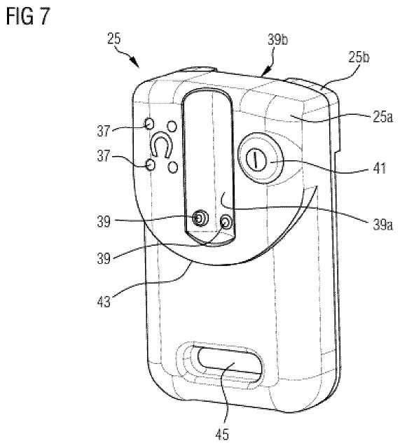

FIG. 7 a perspective view of the electronic hoof component;

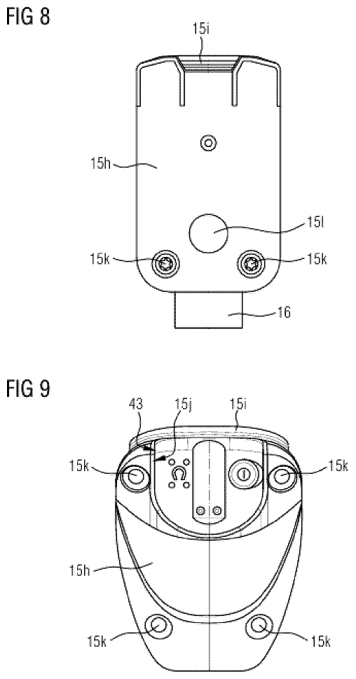

FIG. 8 a rear view of the hoof component holder;

FIG. 9 a perspective top view of the electronic hoof component in the hoof component holder; and

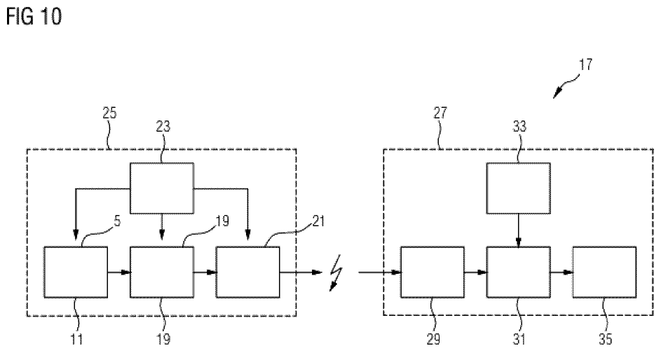

FIG. 10 a schematic diagram of an embodiment of the reaction force measuring system according to the invention, in the form of a block diagram.

The above figures are described in Cartesian coordinates with a longitudinal direction X, a transverse direction Y perpendicular to the longitudinal direction X, and a vertical direction Z perpendicular to both the longitudinal direction X and the transverse direction Y. The longitudinal direction X can also be referred to as depth X, the transverse direction Y as width Y, and the vertical direction Z as height Z. The longitudinal direction X and the transverse direction Y together form the horizontal X, Y, which can also be referred to as the horizontal plane X, Y. The longitudinal direction X, the transverse direction Y, and the vertical direction Z can also be referred to collectively as spatial directions X, Y, Z or as Cartesian spatial directions X, Y, Z.

FIG. 1 shows, in a perspective view from below, the structure of an exemplary reaction force measuring plate 1 as sensor system 1 with a closed horseshoe-shaped rigid carrier plate 3, which has a first surface 3a and a second surface 3b. Seven resistive force measurement sensors 5 with a matching rectangular basic shape are attached to the first surface 3a at equal distances from each other. The free surface of each of the force measurement sensors 5 faces downwards in the direction of a horse's hoof, see FIG. 4, and thus away from the ground (not shown). The force measurement sensors 5 can also be referred to as force sensors 5 or force sensor elements 5.

The force measurement sensors 5 are integrally implemented on a sensor carrier film 9, which also carries sensor signal lines as conductors (not shown) for connecting each sensor. The sensor carrier film 9, together with the force measurement sensors 5 formed thereon, can be manufactured using conventional means of printed circuit board technology, which are known to those skilled in the art. The sensor carrier film 9 is applied with its rear side to the first surface 3a of the carrier 3. This is done using an adhesive and/or an adhesive layer, such as double-sided adhesive tape.

There is also an elastomeric force transmission surface element 11 which, like the sensor carrier film 9, corresponds approximately to the surface area of the rigid carrier plate 3. The elastomeric force transmission surface element 11 can also be referred to as an elastomeric pressure conduit element 11. The elastomeric force transmission surface element 11 has a carrier film 15 facing the rigid carrier plate 3 or the sensor carrier film 9, which is connected to the force transmission surface element 11 by vulcanization and also corresponds approximately to the area of the rigid carrier plate 3. The force transmission surface element 11 is also bonded to the sensor carrier film 9 by means of the carrier film 15, which is facilitated or achieved by the corresponding combination of materials. At the same time, the vulcanized bond between the carrier film 15 and the force transmission surface element 11 allows an elastomeric material to be used for the force transmission surface element 11 and still be bonded to the sensor carrier film 9 by means of the carrier film 15.

Several elastomeric force transmission studs 7, which can also be referred to as elastomeric pressure conduit studs 7, are formed on the elastomeric force transmission surface element 11, which point toward the substrate or away from the force measurement sensors 5 or the sensor carrier film 9. Each force measurement sensor 5 is assigned exactly one force transmission stud 7, so that the force measurement sensor 5 and its force transmission stud 7 are designed to be congruent and positioned relative to each other. This positioning is ensured by the fact that the force transmission studs 7 are integrally formed with the elastomeric force transmission surface element 11.

A rigid load distribution plate 13 is arranged along the vertical axis Z away from the force transmission surface element 11 and parallel to it in the horizontal X, Y, which rests against the force transmission studs 7. Thus, via the force transmission studs 7, a force can be transmitted from the ground to the rigid load distribution plate 13, whereby the loads can act on the force measurement sensors 5 without force shunts. The rigid load distribution plate 13 can be implemented as an organic sheet metal made of polyamide with glass fiber reinforcement.

According to the invention, the reaction force measuring plate 1 is arranged and/or integrated within the interior space (not designated) of a hoof boot 15, which is made of an elastomeric material, encloses the carrier 3 in a planar, parallel manner on this underside as a hoof boot sole 15a, see FIG. 3, and further in the vertical direction Z from the edge and/or from the border of the carrier 3 via the sensor carrier film 9, the force transmission surface element 11 and the rigid load distribution plate 13 at the edge side upwards (not shown in FIG. 3). The hoof boot sole 15a has a sole profile 15b facing downwards away from the reaction force measuring plate 1. The latter area represents a hoof boot wall 15c, see FIG. 4. This closes off the sensor carrier film 9, including the force measurement sensors 5, and the force transmission surface element 11, including the force transmission studs 7, to the outside and thus protects them from external influences.

Furthermore, an inner protective element 14 is provided, which adjoins the inside of the hoof boot wall 15c in the opposite direction by means of a seal 14a in the form of a sealing lip 14a and encloses and protects the carrier 3 on the back as described above.

The force measurement sensors 5 are connected via printed sensor signal lines and can be read out, as will be described in more detail below. For this purpose, a connector element 16 in the form of a plug outlet 16 is provided, which ends in a plug 16a. The plug outlet 16 and an internal part of the plug 16a are enclosed by a cable guard 16b, thereby protecting them from external influences.

The plug outlet 16 is guided out through a connector element opening 15d in the form of a cable opening 15d and is otherwise completely closed by a connection element clamp 15e in the form of a cable clamp 15e in a spring-elastic manner.

An electronic hoof component 25, which will be described in more detail with reference to FIGS. 7 to 10, is fixedly arranged on the outside of the hoof boot 15 and is connected to the force measurement sensors 5 by means of the plug outlet 16, facing outwards from the other hoof (not shown) in the transverse direction Y. The electronic hoof component 25 is essentially enclosed on the outside by a housing which is formed by a front housing half 25a facing away from the hoof boot wall 15c and a rear housing half 25b facing the hoof boot wall 15c. The hoof component holder 15h is formed in one piece. The hoof component holder 15h receives the electronic hoof component 25 along a joining direction, as described in more detail below. Then, the hoof component holder 15h together with the accommodated electronic hoof component 25 is fixedly connected to the hoof boot 15 or to its hoof boot wall 15c by means of screw connections 15k or rivets (not shown).

The electronic hoof component 25 has four display elements 37 in the form of light conductors 37, which are arranged and illuminated around a symbol of a horseshoe, which symbolizes the orientation of the hooves of the hoofed animal, so that the hoof on which the respective hoof boot system 15, 25 is used can be displayed.

The electronic hoof component 25 also has an operating element 41 in the form of an on/off switch 41 for switching the electronic hoof component 25 on and off.

The electronic hoof component 25 further comprises a charging port 39 in the form of a pair of charging contacts 39, which serve to charge an electrical energy storage device (not shown) belonging to the electronic hoof component 25.

As already mentioned, the electronic hoof component 25 is inserted from above into the hoof component holder 15h and held there in a positive-fitting and force-fitting manner. A gap (not shown) forming on the inside between the hoof component holder 15h and the rear housing half 25b is closed from above, along the joining direction by an elastically foldable protective edge 15i in order to protect the gap between the electronic hoof component 25 that is accommodated and the hoof component holder 15h from contamination.

Furthermore, the hoof component holder 15h forms a pressure compensation element 15l facing the rear housing half 25b. The pressure compensation element 15l is designed as a semi-permeable membrane, which allows the exchanging of air and is simultaneously waterproof.

Furthermore, a spring-elastic protrusion (not shown) of the hoof component holder 15h engages into a corresponding pull-out safety device 45 of the front housing half 25a of the electronic hoof component 25, which can press perpendicular to the joining direction onto the rear housing half 25b of the electronic hoof component 25 and thereby bring about a positive hold, so that a sufficiently secure hold can be achieved, even in the event of an intense movement.

At the same time, the front housing half 25a of the electronic hoof component 25 and the hoof component holder 15h form corresponding stop edges 15j, 43 as collars 15j, 43, which abut or rest against each other and thus improve the hold, and also seal against contamination. This ensures a firm hold of the hoof component holder 15h and the electronic hoof component 25 in order to be firmly connected to the hoof boot 15 or to its hoof boot wall 15c by screwing or riveting. The protective edge 15i is designed to be elastically foldable upwards in order to be able to reach the charging contacts 39 by means of a bow-shaped charging clip (not shown).

The hoof component holder 15h is surrounded in a U-shape at the bottom as well as laterally, forwards and backwards by a hoof component guard 15f. The hoof boot 15 can be opened and closed using a hoof boot closure 15g in order to attach or remove the hoof boot 15 to the hoof.

FIG. 10 is a schematic diagram of a reaction force measuring system 17, which can be formed, especially with a reaction force measuring plate according to one of FIGS. 1 to 4. The representation takes the form of a functional block diagram and is not intended to show the exact design of the system components.

In addition to the force measurement sensors 5 and their sensor signal lines shown in FIGS. 1 to 4, the reaction force measuring system 17 also comprises a sensor signal pre-processing unit 19, which is connected to the force measurement sensors 5 via the sensor signal lines and serves to perform preprocessing and formatting of the sensor signals that is advantageous for external signal transmission. On the output side, the sensor signal pre-processing unit 19 is connected to a wireless sensor signal transmitter 21, which can operate according to the Bluetooth standard, for example, but also according to another standard for wireless message transmission, depending on the application. The above-mentioned components are assigned an energy source 23, which in the simplest case is a commercially available primary cell or a rechargeable battery. If a rechargeable battery is used, it can be assigned a charging socket (not shown in the figure) for recharging when installed.

All of the above-mentioned components are advantageously arranged in the electronic hoof component 25.

When the system is in use, the electronic hoof component 25 is in wireless signal connection via the sensor signal transmitter 21 with a sensor signal receiver, evaluation and display device 27, which is placed away from the animal to be examined. The device 27 comprises a wireless sensor signal receiver 29 which is configured to communicate with the sensor signal transmitter 21 on the electronic hoof component 25 and which feeds the received signals to a signal evaluation unit 31, where they are subjected to evaluation according to a program stored in a program memory 33.

Finally, a display unit 35 serves to display the evaluation results, for example for a therapist or trainer. The components of the sensor signal receiver, evaluation and display device can be implemented, for example, in a notebook, tablet or smartphone with a suitable evaluation app.

### REFERENCE LIST (PART OF THE DESCRIPTION)

X longitudinal direction; depth
Y transverse direction; width
Z vertical direction; height
X, Y horizontal; horizontal plane
1 sensor system, reaction force measuring plate
3 carrier plate
3a first surface
3b second surface
5 force measurement sensor
7 (elastomeric) elastic force transmission studs; (elastomeric) elastic pressure conduit studs
9 sensor carrier film
11 (elastomeric) force transmission surface element; (elastomeric) pressure conduit element
13 load distribution plate
14 inner protective element
14a seal, sealing lip
15 hoof boot; outer protective element
15a hoof boot sole
15b sole profile
15c hoof boot wall
15d connector element opening; cable opening
15e connector element clamp; cable clamp
15f hoof component guard
15g hoof boot closure
15h hoof component holder
15i protective edge of the hoof component holder 15h
15j stop edge and/or collar of the hoof component holder 15h
15k screw connections of the hoof component holder 15h
15l pressure compensation element of the hoof component holder 15h
16 connector element; plug outlet; cable outlet
16a plug
16b cable guard
17 reaction force measuring system
19 sensor signal pre-processing unit
21 wireless sensor signal transmitter
23 energy source
25 electronic hoof component
25a front housing half
25b rear housing half
27 sensor signal, sensor signal receiver, evaluation and display device
29 wireless sensor signal receiver
31 signal evaluation unit
33 program memory
35 display unit
37 display elements; light conductor
39 charging port; charging contacts
41 operating element; on/off switch
43 stop edge or collar of the electronic hoof component 25
45 pull-out protection device

1. A hoof boot system with
a hoof boot with
at least one sensor system and
a hoof boot wall with a hoof component holder and with
an electronic hoof component which is held by the hoof component holder and is designed to read the sensor system of the hoof boot.

2. The hoof boot system according to claim 1,
wherein the electronic hoof component is accommodated and held in a securing system, preferably in an interior space of the hoof component holder,
wherein the hoof component holder preferably has a protective edge, which can preferably be folded up elastically, in order to protect a gap between the accommodated electronic hoof component and the hoof component holder from contamination.

3. The hoof boot system according to claim 1, wherein the hoof component holder has a stop edge along the joining direction and the electronic hoof component has a corresponding stop edge opposed along the joining direction.

4. The hoof boot system according to claim 1, wherein the hoof component holder has a pressure compensation element perpendicular to the joining direction and/or the electronic hoof component has a pull-out protection device opposed perpendicular to the joining direction.

5. The hoof boot system according to claim 1,
wherein the electronic hoof component has at least one charging port, preferably a pair of charging contacts.

6. The hoof boot system according to claim 1,
wherein the electronic hoof component has an operating element, preferably an on/off switch, which is accessible from the outside, preferably laterally and facing away from the hoof during use.

7. The hoof boot system according to claim 1,
wherein the electronic hoof component has at least one display element visible from the outside, preferably laterally and facing away from the hoof during use.

8. The hoof boot system according to claim 1, wherein the electronic hoof component has four display elements that are visible from the outside, preferably laterally and facing away from the hoof during use, said display elements being arranged and configured to display the respective hoof during use.

9. The hoof boot system according to claim 1,
wherein the hoof boot has a hoof boot sole which completely encloses the sensor system relative to the ground, and
wherein the hoof boot wall is integrally formed with the hoof boot sole and encloses the sensor system and, during use, the hoof, at least partially, preferably at least substantially,
wherein the hoof boot wall preferably has a sole profile facing away from the carrier plate.

10. The hoof boot system according to claim 1,
wherein the electronic hoof component is electrically and conductively connected to the sensor system of the reaction force measuring plate by means of a connector element,
wherein the connector element passes through a connector element opening of the hoof boot wall, preferably directly above the hoof boot sole and/or laterally.

11. The hoof boot system according to claim 10,
wherein the connector element opening is otherwise at least partially, preferably completely, closed by a connector element clamp.

12. The hoof boot system according to claim 1,
the electronic hoof component is arranged on the outside of the hoof boot wall,
wherein the hoof boot wall has a hoof component guard which surrounds the electronic hoof component at least from below, preferably further at least laterally on one side, particularly preferably laterally on both sides.

13. The hoof boot system according to claim 1,
wherein the sensor system is a reaction force measuring plate for detecting the ground reaction force distribution over the ground contact surface of the foot of a hoofed animal when it strikes the ground, with
a preferably rigid carrier plate with a first surface that faces the hoof in use and an opposite second surface facing the ground, or vice versa, a plurality of flat force measurement sensors fixed in position on the first surface of the carrier plate,
a plurality, especially a plurality corresponding to the plurality of force measurement sensors, of elastic force transmission studs which are fixed to the free surfaces of the force measurement sensors.

14. A hoof boot for use in a hoof boot system according to claim 1.

15. An electronic hoof component for use in a hoof boot system according to claim 1.
